(19) United States
(12) Patent Application Publication
(10) Pub. No.: US 2019/0274748 A1
(43) Pub. Date: Sep. 12, 2019

(54) MEASURING IMPEDANCE FOR ELECTROSURGICAL TOOLS

(71) Applicant: Ethicon LLC, Guaynabo, PR (US)

(72) Inventors: Benjamin J. Danziger, Cincinnati, OH (US); David C. Yates, West Chester, OH (US); Jeffrey L. Aldridge, Lebanon, OH (US); Andrew Carroll, Cincinnati, OH (US); Eitan Wiener, Cincinnati, OH (US); Cameron R. Nott, Loveland, OH (US)

(21) Appl. No.: 15/913,389

(22) Filed: Mar. 6, 2018

(51) Int. Cl. A61B 18/12; A61B 18/14

(52) U.S. Cl. CPC A61B 18/1206; A61B 2018/00702; A61B 18/1445

(57) ABSTRACT

Methods, devices, and systems for measuring impedance for electrosurgical tools are provided. In general, a surgical device configured to apply energy to tissue can be configured to deliver the energy to the tissue based on an impedance of the tissue. The impedance can be determined based on an electrical signal transmitted to the tissue via the surgical device, such as by one or more electrodes of the surgical device that are in contact with the tissue. The impedance determination can include determining both real and imaginary components of the impedance, thereby allowing a phase shift to be identified and for an actual impedance to be determined by correcting for the phase shift. The actual impedance of the tissue can be used to identify a type of the tissue.

### FIELD

The present disclosure relates generally to measuring impedance for electrosurgical tools.

### BACKGROUND

Surgical devices are used in various open, endoscopic, and laparoscopic surgeries to manipulate tissue, staple tissue, and/or transect tissue volumes and blood vessels. These devices can include jaws for grasping tissue therebetween and, in at least some device, a cutting mechanism that can be advanced through the grasped tissue to transect the tissue. The cutting mechanism can be designed to travel within a track formed in one or both jaws. The devices can also be used to seal tissue volumes and blood vessels being transected, for instance by applying electrical energy to the grasped tissue to seal it before tissue transection is completed. For example, various mono-polar and bi-polar radio frequency (RF) surgical instruments and surgical techniques have been developed for sealing tissue volumes and blood vessels. Electrodes can be disposed on a face of one or both of the jaws and can apply energy to the grasped tissue to promote hemostasis.

For surgical devices that apply energy to tissue, it can be difficult for the correct amount of energy to be applied to the tissue. At least some of these surgical devices connect to a generator that provides energy thereto for delivery to tissue grasped by the surgical device. However, due to factors such as the type of tissue being energized, design of the surgical device, and effects of cables (e.g., inductance and length) through which energy is delivered from the generator to the surgical device and/or through the surgical device to the tissue, it can be difficult for the correct amount of energy to be applied to the tissue to promote hemostasis without causing one or more undesirable effects such as overheating of the tissue, underheating of the tissue, and/or lengthy duration of energy application. Knowing the impedance of the tissue grasped by the surgical device may help the generator electronically determine the correct amount of energy to be applied to the tissue, but it can be difficult to measure an accurate impedance of the tissue. Thus, even if impedance of the tissue could be measured, an incorrect amount of energy may still be applied to the tissue because of inaccuracy of the impedance measurement.

Accordingly, there remains a need for improved measuring impedance for electrosurgical tools.

### SUMMARY

In general, methods, devices, and systems for measuring impedance for electrosurgical tools are provided.

In one aspect, a surgical system is provided that in one embodiment includes a generator configured to transmit a signal along first, second, third, and fourth wires to an end effector of a surgical device in contact with matter within a body of a patient, receive a return signal from each of the first, second, third, and fourth wires, analyze the return signals to determine an impedance of the matter, and determine, based on the determined impedance, an algorithm for application of energy from the generator to the matter via an electrode at the end effector of the surgical device in contact with the matter.

The surgical system can have any number of variations. For example, the matter can be tissue, and the generator can be configured to apply the energy to the matter using the determined algorithm. For another example, the generator can be configured to determine, based on the determined impedance, that an error condition is present, the generator can be configured to cause an alert to be provided to a user that indicates the presence of the error condition, and the error condition can be one of a short circuit, an open circuit, and the determined impedance indicating that the matter is not tissue. For yet another example, determining the impedance of the matter can include determining a real component of the impedance and an imaginary component of the impedance to determine a phase shift, and using the determined phase shift to identify actual impedance of the matter. For still another example, the matter can be tissue, determining the impedance of the matter can indicate a type of the tissue, and determining the algorithm can include selecting one of a plurality of predetermined algorithms based on the type of the tissue. For another example, the matter can be tissue, determining the impedance of the matter can indicate a type of the tissue, and determining the algorithm can include dynamically generating an algorithm based on the type of the tissue. For still another example, the transmitted signal can be an excitation signal including a positive supply voltage along the first and third wires and a negative supply voltage along the second and fourth wires. For another example, the transmitted signal can be a drive signal having a frequency in a range of about 500 to 2000 Hz. For yet another example, the system can further include the surgical device.

In another embodiment, a surgical system includes a surgical device including a proximal handle portion having an elongate shaft extending distally therefrom. The elongate shaft has an end effector at a distal end thereof. The end effector includes an electrode configured to contact tissue and apply energy thereto. The surgical device is configured to receive from a generator a first signal along first, second, third, and fourth wires that extend to the end effector. The surgical device is also configured to, in response to receiving the first signal, return a second signal to the generator along the first, second, third, and fourth wires, and, after returning the second signal, receive a third signal from the generator and in response thereto apply the energy via the electrode.

The surgical system can vary in any number of ways. For example, the surgical system can further include the generator, and the generator can be configured to receive the second signal and in response thereto determine an impedance of the tissue contacted by the electrode, and the third signal can be determined in real time by the generator based on the determined impedance.

In another aspect, a surgical method is provided that in one embodiment includes transmitting a signal along each of first, second, third, and fourth wires from a generator to a distal end of a surgical device in contact with matter within a body of a patient, receiving at the generator a return signal from each of the first, second, third, and fourth wires, analyzing the return signals to determine an impedance of the matter, and determining, based on the determined impedance, an algorithm for application of energy from the generator to the matter via an electrode at the distal end of the surgical device in contact with the matter.

The surgical method can vary in any number of ways. For example, the surgical method can further include applying the energy from the generator to the matter using the determined algorithm, and the matter can be tissue. For another example, the surgical method can further include determining, based on the determined impedance, that an error condition is present, the surgical method can further include causing an alert to be provided to a user that indicates the presence of the error condition, and the error condition can be one of a short circuit, an open circuit, and the determined impedance indicating that the matter is not tissue. For yet another example, determining the impedance of the matter can include determining a real component of the impedance and an imaginary component of the impedance to determine a phase shift, and using the determined phase shift to identify actual impedance of the matter. For still another example, the matter can be tissue, determining the impedance of the matter can indicate a type of the tissue, and determining the algorithm can include selecting one of a plurality of predetermined algorithms based on the type of the tissue. For another example, the matter can be tissue, determining the impedance of the matter can indicate a type of the tissue, and determining the algorithm can include dynamically generating an algorithm based on the type of the tissue. For still another example, the transmitted signal can be an excitation signal including a positive supply voltage along the first and third wires and a negative supply voltage along the second and fourth wires. For yet another example, the transmitted signal can be a drive signal having a frequency in a range of about 500 to 2000 Hz. For another example, the surgical method can further include removably and replaceably connecting the surgical device to the generator.

### BRIEF DESCRIPTION OF DRAWINGS

This invention will be more fully understood from the following detailed description taken in conjunction with the accompanying drawings, in which:

FIG. 1 is a side schematic view of one embodiment of a surgical device;

FIG. 2 is a side, partially transparent schematic view of the surgical device of FIG. 1;

FIG. 3 is a perspective, partially cross-sectional and transparent schematic view of a distal portion of the surgical device of FIG. 1;

FIG. 3A is a schematic view of one embodiment of a jaw of a surgical device that includes a plurality of electrodes;

FIG. 3B is a schematic view of another embodiment of a jaw of a surgical device that includes a plurality of electrodes;

FIG. 3C is a schematic view of yet another embodiment of a jaw of a surgical device that includes a plurality of electrodes;

FIG. 3D is a perspective view of one embodiment of an upper jaw of a surgical device that includes a plurality of electrodes;

FIG. 3E is another perspective view of the jaw of FIG. 3D, the jaw being movably coupled to an elongate shaft of the surgical device;

FIG. 3F is another perspective view of the jaw of FIG. 3E;

FIG. 4 is a perspective schematic view of a compression member of the surgical device of FIG. 1;

FIG. 5 is a schematic view of a generator of the surgical device of FIG. 2;

FIG. 6 is a circuit diagram;

FIG. 7 is a flowchart of one embodiment of a method of using the surgical device of FIG. 1;

FIG. 8 is a boxplot showing phase angles of different size vessels at various frequencies;

FIG. 9 is a boxplot showing magnitude of the different size vessels of FIG. 8 at various frequencies;

FIG. 10 is a graph plotting imaginary impedance versus real impedance; and

FIG. 11 is a graph plotting harmonic current versus tissue impedance.

### DETAILED DESCRIPTION

Certain exemplary embodiments will now be described to provide an overall understanding of the principles of the structure, function, manufacture, and use of the devices, systems, and methods disclosed herein. One or more examples of these embodiments are illustrated in the accompanying drawings. Those skilled in the art will understand that the devices, systems, and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

Further, in the present disclosure, like-named components of the embodiments generally have similar features, and thus within a particular embodiment each feature of each like-named component is not necessarily fully elaborated upon. Additionally, to the extent that linear or circular dimensions are used in the description of the disclosed systems, devices, and methods, such dimensions are not intended to limit the types of shapes that can be used in conjunction with such systems, devices, and methods. A person skilled in the art will recognize that an equivalent to such linear and circular dimensions can easily be determined for any geometric shape. Sizes and shapes of the systems and devices, and the components thereof, can depend at least on the anatomy of the subject in which the systems and devices will be used, the size and shape of components with which the systems and devices will be used, and the methods and procedures in which the systems and devices will be used.

Methods, devices, and systems for measuring impedance for electrosurgical tools are provided. In general, a surgical device configured to apply energy to tissue, e.g., an “electrosurgical tool,” can be configured to deliver the energy to the tissue based on an impedance of the tissue. The impedance can be determined based on an electrical signal transmitted to the tissue via the surgical device, such as by one or more electrodes of the surgical device that are in contact with the tissue. The impedance determination can include determining both real and imaginary components of the impedance, thereby allowing a phase shift to be identified and for an actual impedance to be determined by correcting for the phase shift. The phase shift can be present for one or more reasons, such as a type of the tissue, design of the surgical device, and effects of cables used to carry the electrical signal (e.g., different inductances and/or lengths of the cables). Radiofrequency (RF) and other electrical noise does not affect the impedance calculation because the electrical signals are communicated digitally. Determining the actual impedance of the tissue may allow for appropriate energy to be delivered to the tissue to seal the tissue without overheating the tissue, without underheating the tissue, and without taking an undesirably long amount of time to heat the tissue. The actual impedance of the tissue can be used to identify a type of the tissue, as tissue type correlates to impedance. Appropriate energies for promoting hemostasis for different tissue types are known such that identifying the tissue type can allow the appropriate energy to be selected and applied, which may allow for improved hemostasis and/or faster sealing.

In most instances, matter contacted by the surgical device's one or more electrodes will be tissue, in which case energy may be safely applied thereto via the electrode(s). In other instances, matter contacted by the surgical device's one or more electrodes will not be tissue, such as if the surgical device grasps a metal surgical tool at a surgical site instead of tissue, if the surgical device grasps other non-tissue matter at the surgical site such as suture material, staples or other fasteners applied to tissue, gauze, etc., or if jaws of the surgical device fail to grasp tissue or anything else therebetween such that facing surfaces of the jaws contact or nearly contact each other. In such instances energy should not be applied, e.g., to prevent burning of the matter contacted by the electrode(s), to prevent harming the patient, because there is a short circuit or an open circuit, etc. A surgeon or other medical personnel may have intended tissue to be in contact with the electrode(s) for energy application but not be aware that the electrode(s) are contacting matter to which energy should not be applied, e.g., due to obstructed or otherwise impaired visualization of the surgical site. Determining the impedance of the matter contacted by the one or more electrodes may allow for determination of a situation where energy should not be applied via the electrode(s) because the determined impedance does not correlate to known tissue impedance values and thus indicates an error condition such an non-tissue matter being in contact with the electrode(s), a short circuit, or an open circuit. For example, impedance of metal or fabric would not correlate to known tissue impedance values. An alert can be provided in response to the identification of the error condition, such as a visual, auditory, and/or tactile warning to a user (e.g., a surgeon and/or other medical personnel) provided via a speaker, display screen, and/or other device, to inform a user and/or help prevent the energy from being applied and causing harm.

In an exemplary embodiment, the electrical signal transmitted to the tissue via the surgical device is transmitted along four wires. Using four wires may facilitate determining both real and imaginary components of the impedance. In general, transmitting a signal along each of the four wires may allow for variations in return signals from each of the wires to be determined, thereby allowing for an accurate impedance measurement using the return signals. For example, a first wire can carry a positive supply voltage (V+) of an output drive signal, a second wire can carry a negative supply voltage (V−) of the output drive signal, a third wire can carry a positive supply voltage (V+) of a power out signal, and a fourth wire can carry a negative supply voltage (V−) of the power out signal. Under ideal conditions the V+ of the output drive signal and V+ of the power out signal would be equal to one another, and the V− of the output drive signal and V− of the power out signal would be equal to one another. However, under actual conditions in which various error(s) are introduced, e.g., due to wire and/or cable effects, etc., the V+ values and/or the V− values are not equal. By determining phase shift of the V+ signals and the V− signals, an actual impedance can be more accurately determined than if less than four wires are used. For another example, each of the four wires can carry a signal having a different frequency. Each of the frequencies can be a low frequency in a range of about 500 to 2000 Hz. A person skilled in the art will appreciate that a value may not be precisely at a certain value but nevertheless be considered to be at about that value due to any number of factors, such as sensitivity of measurement equipment and manufacturing tolerance. Tissue impedances equal to or less than about 3000 Ohms can be measured using the low frequencies.

In an exemplary embodiment, the surgical device can be configured to removably and replaceably connect to a generator configured to supply energy to the surgical device to be applied to tissue. The generator can also be configured to supply the electrical signal to the surgical device, to receive a return signal from the surgical device in response to the supplied electrical signal, to determine the impedance using the return signal, and to supply the energy to the surgical device based on the determined impedance. The generator being configured to determine the impedance may allow the surgical device to lack processing capabilities, which may help reduce an overall cost of the device and/or facilitate disposability and/or cleaning of the surgical device. The generator being configured to determine the impedance may facilitate upgrades to processing components (e.g., processor, memory, etc.) and/or upgrades to energy application algorithms, which are discussed further below, since upgrades need only be performed at the generator instead of on each individual surgical device that connects therewith. Because the generator's output, e.g., the supplied electrical signal, is a function of measured impedance, the impedance being more accurately measured using four wires may therefore improve the accuracy of the output, improve the accuracy of tissue treatment algorithms, and/or allow for the sensing abnormal conditions as discussed further below. Various embodiments of using measured impedance to treat tissue that may be improved upon by the methods, systems, and devices of the present application that may more accurately measure impedance are described in U.S. Pat. No. 5,558,671 entitled “Impedance Feedback Monitor For Electrosurgical Instrument” filed Sep. 23, 1994, U.S. Pat. No. 5,817,093 entitled “Impedance Feedback Monitor With Query Electrode For Electrosurgical Instrument” filed Oct. 6, 1998, and U.S. Pat. No. 9,060,776 entitled “Surgical Generator For Ultrasonic And Electrosurgical Devices” filed Oct. 1, 2010, which are hereby incorporated by reference in their entireties.

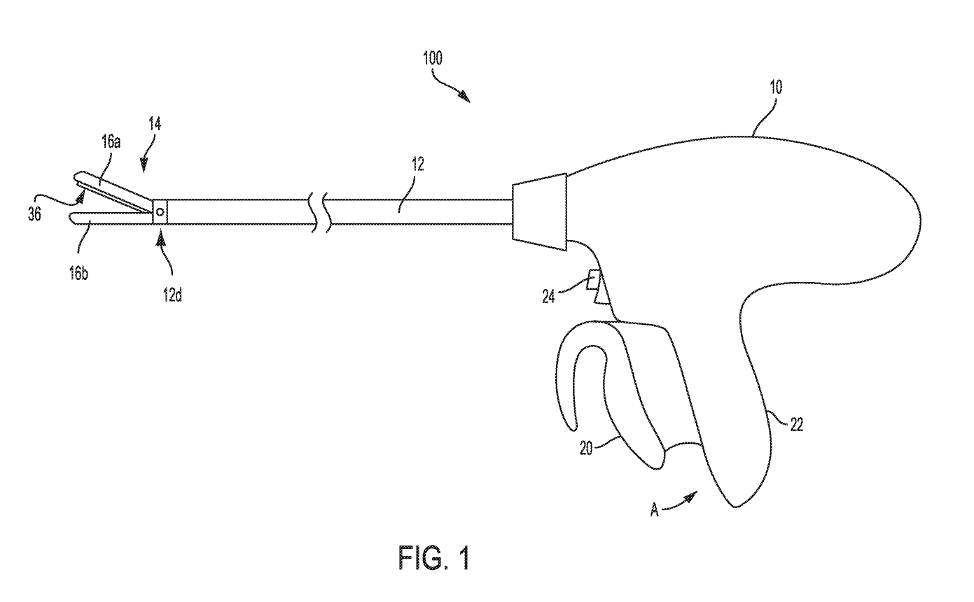

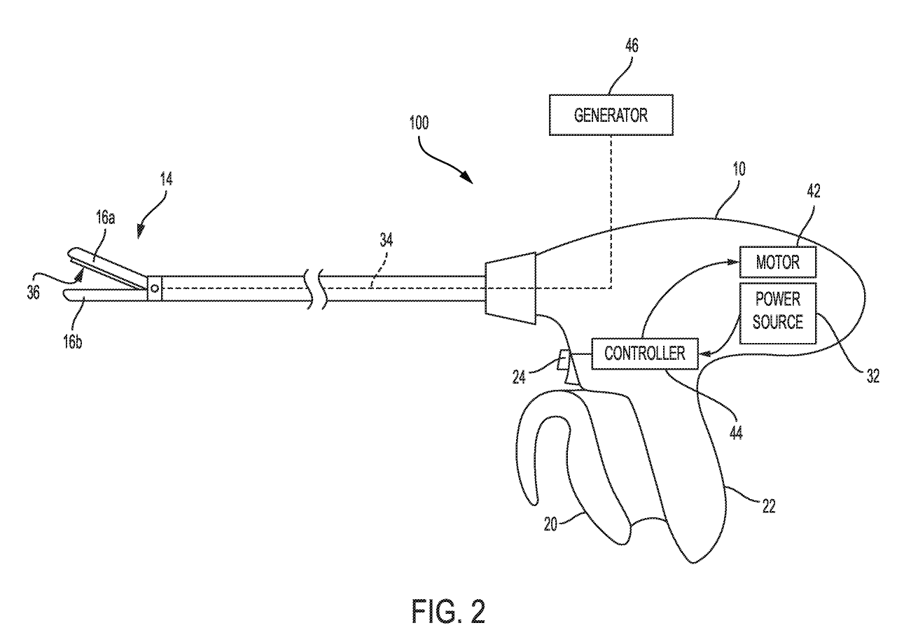

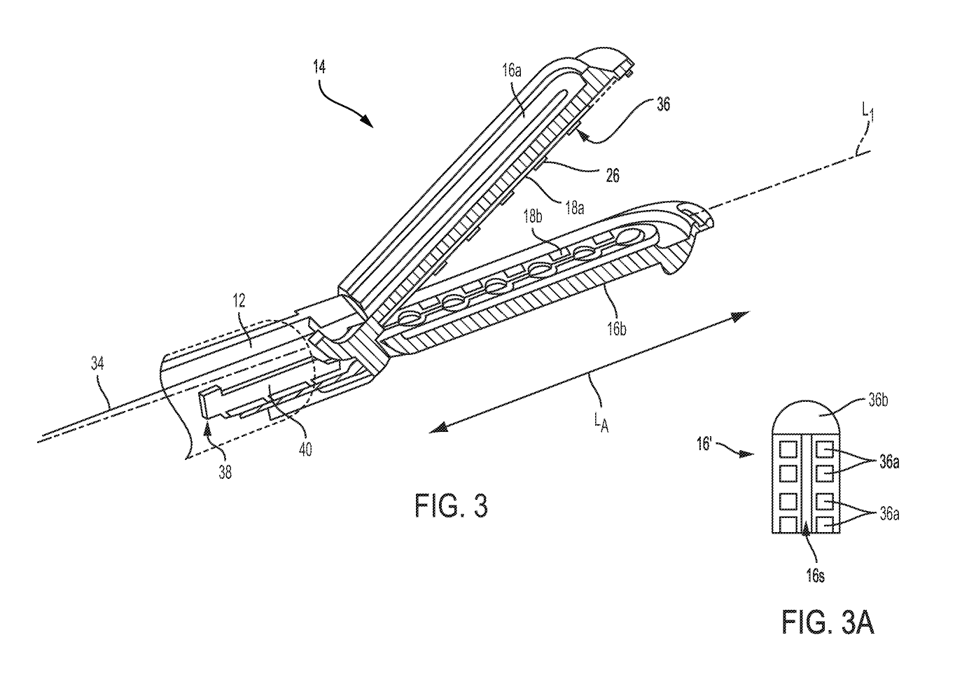

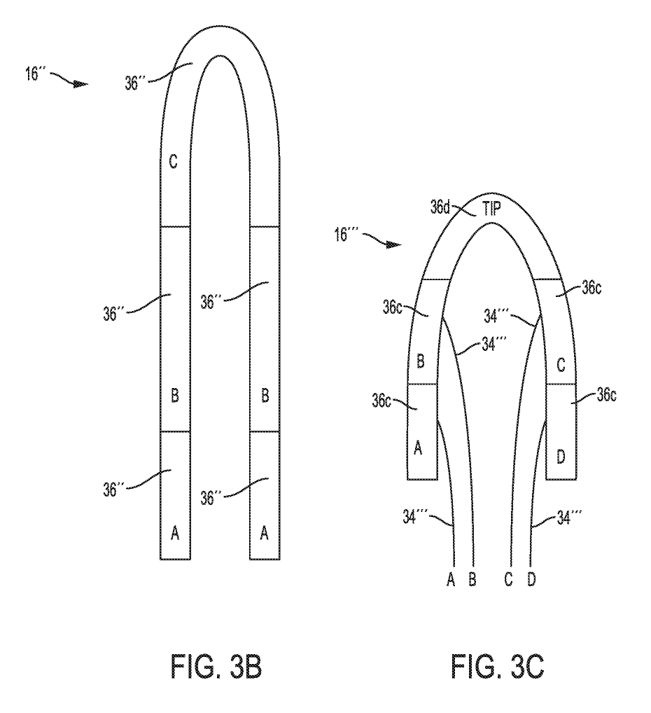

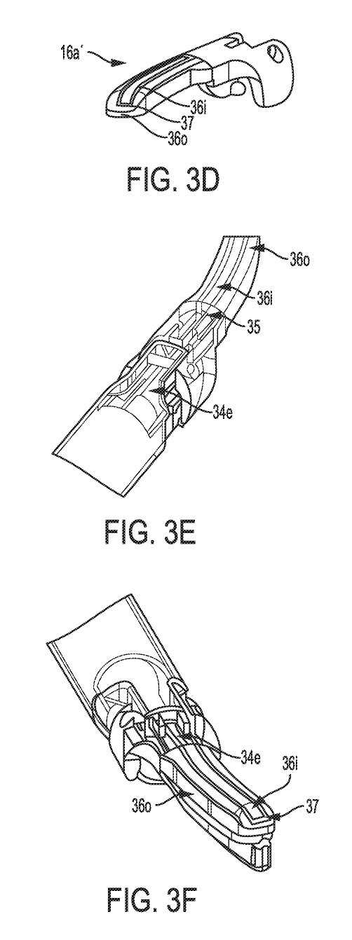

FIGS. 1 and 2 illustrate one exemplary embodiment of a surgical device 100 including an elongate shaft 12 having an end effector 14 at a distal end 12d thereof. A distal portion of the device 100 is illustrated in FIG. 3. The shaft 12 extends distally from a housing or proximal handle portion 10 of the surgical device 100. The shaft 12 can be removably and replaceably attached to the housing 10 or components therein in manners that are known to those skilled in the art. In other embodiments, the shaft 12 can be integrally formed with the housing 10.

The housing 10 can be any type of pistol-grip or other type of handle that is configured to carry and/or engage various components used in conjunction with actuating the end effector 14, such as motors, controllers, levers, triggers, sliders, and/or other components, and/or with performing other surgical functions or movements of the device 100. The housing 10 includes a closure actuator 20 and includes a stationary arm 22, also referred to herein as a stationary handle. In general, the closure actuator 20 is configured to be actuated, e.g., moved relative to the stationary arm 22, to control opening and closing of the end effector 14. A person skilled in the art will appreciate that while the term “handle” can be used in conjunction with the stationary arm 22, in some embodiments, such as those that involve actuation of the closure actuator by a robotic surgical system, electronic system, or other controlled system and thus do not involve manual actuation of the closure actuator, the stationary arm 22 does not have to be “handled” by hand. Thus, the stationary arm 22 can serve as a reference point to describe the location of the closure actuator 20, and does not have to be “handled” by hand. Similarly, the handle portion 10 need not be “handled” by hand.

In some embodiments, the housing 10 can be configured for use with a robotic surgery platform, as opposed to a user's hand. In such embodiments, the closure actuator 20 can have a different configuration than shown in the embodiment of FIGS. 1 and 2, such as by being included as part of a tool housing configured to be operatively coupled to the robotic surgery platform to allow the robotic surgery platform to provide inputs to the tool housing to selectively open and close the end effector 14, e.g., to provide an input to the tool housing to cause linear movement of a rod or other force-translating component of the surgical device. Various embodiments of tool housings of surgical instruments configured to be operatively coupled to a robotic surgery platform are further described in International Pat. Pub. No. WO 2014/151952 entitled “Compact Robotic Wrist” filed Mar. 13, 2014, International Pat. Pub. No. WO 2014/151621 entitled “Hyperdexterous Surgical System” filed Mar. 13, 2014, U.S. patent application Ser. No. 15/200,283 entitled “Methods, Systems, And Devices For Initializing A Surgical Tool” filed Jul. 1, 2016, and in U.S. patent application Ser. No. 15/237,653 entitled “Methods, Systems, And Devices For Controlling A Motor Of A Robotic Surgical System” filed Aug. 16, 2016, which are hereby incorporated by reference in their entireties.

Referring again to FIGS. 1 and 2, movement of the closure actuator 20 relative to the stationary arm 22, or between uncompressed (open) and compressed (closed) configurations or positions, can be configured to control the movement of the end effector 14. More particularly, as shown in this illustrated embodiment, the end effector 14 includes upper and lower jaws 16a, 16b, which can be opened and closed by moving the closure actuator 20 with respect to the stationary arm 22. As described further below, the jaws 16a, 16b can be configured to grasp tissue, and then additional surgical functions can be performed on the grasped tissue using the device 100 and/or other surgical tools, such as cutting or transecting and/or sealing the tissue. While the illustrated end effector 20 has a pair of opposed jaws 16a, 16b, other types, size, shapes, and configurations of end effectors can be used as an end effector in the surgical devices described herein. A distance between the jaws 16a, 16b is greater when the jaws 16a, 16b are in an open position, which is shown in FIGS. 1 and 2, than when the jaws 16a, 16b are in a closed position. The distance between the jaws 16a, 16b increases as the jaws 16a, 16b move from the closed configuration to and the open configuration, and similarly decreases when the jaws 16a, 16b move from the open configuration to the closed configuration. As in this illustrated embodiment, the end effector 14 can be defaulted in the open position, and actuation of the closure actuator 20 to move the closure actuator 20 in a direction A toward the stationary handle 22 can be configured to close the end effector 14. Subsequent actuation of the closure actuator 20 to move away from the stationary handle 22, e.g., in the direction opposite to the direction A, can be configured to open the end effector 14.

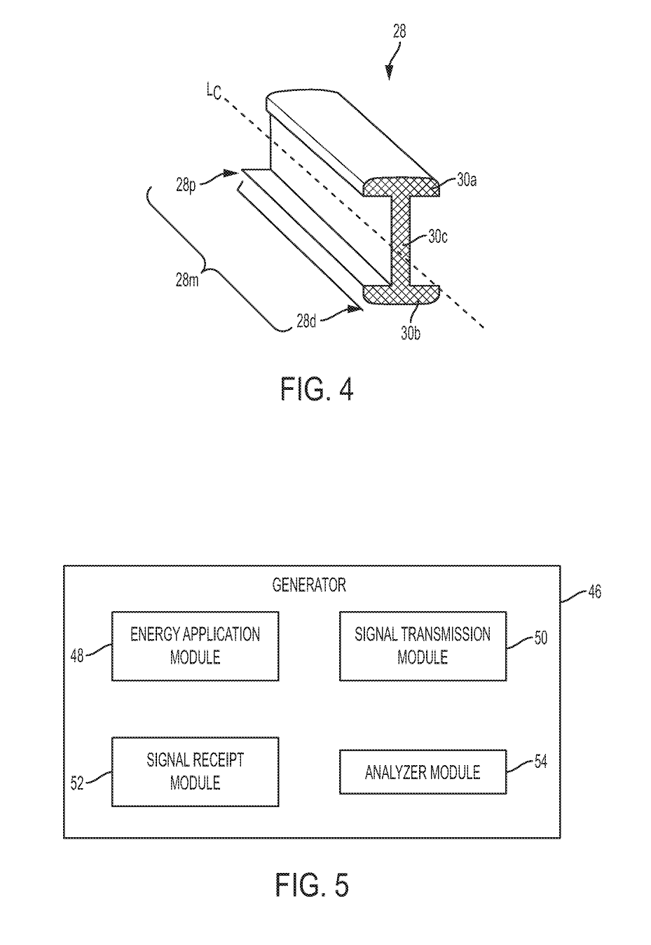

The shaft 12 includes an inner passageway 38 extending longitudinally through the shaft 12 along a longitudinal axis L₁ of the shaft 12. The inner passageway 38 is configured to contain therein one or more mechanisms, such as a drive shaft 40 having a compression member 28 at a distal end thereof, one or more electrical leads 34, etc. The compression member 28 is obscured in FIG. 3 but is illustrated as a standalone element in FIG. 4.

The end effector 14 includes the first, upper jaw 16a and the second, lower jaw 16b, one or both of which can be configured to move about the longitudinal axis L₁ to open and close the end effector 14. Both of the jaws 16a, 16b can be movable relative to the shaft portion 12 or, alternatively, a single one of the jaws 16a, 16b can be configured to pivot so that the end effector 14 can move between closed and open positions. When the jaws 16a, 16b are in the closed position, a longitudinal axis of the upper jaw 16a can be substantially parallel to a longitudinal axis of the lower jaw 16b, and opposed tissue-engaging surfaces 18a, 18b of the jaws 16a, 16b can be in direct contact with one another when tissue is not disposed between the jaws 16a, 16b. Alternatively, the tissue-engaging surfaces 18a, 18b of the jaws 16a, 16b can be spaced a small distance apart from one another when the jaws 16a, 16b are in the closed position, which may facilitate tissue disposed between the jaws 16a, 16b being adequately held by the jaws 16a, 16b when the jaws 16a, 16b are in the closed position. In the embodiment illustrated in FIGS. 1-3, the upper jaw 16a is configured to pivot relative to the shaft 12 and relative to the lower jaw 16b while the lower jaw 16b remains stationary. In other embodiments, the lower jaw 16b can be configured to move with respect to the upper jaw 16a which remains stationary, or both jaws 16a, 16b can be configured to pivot with respect to each other.

The jaws 16a, 16b each have a substantially elongate and straight shape as in this illustrated embodiment, but one or both of the jaws 16a, 16b can have another shape, such as by being curved relative to the longitudinal axis L₁. The jaws 16a, 16b can have any suitable axial length L_{A} for engaging tissue, where the axial length L_{A} is measured along a longitudinal axis L₁ of the end effector 14, as shown in FIG. 3. The axial length L_{A} of the jaws 16a, 16b can be selected based on any number of factors, such as the targeted anatomical structure for transection and/or sealing, the size, shape, and configuration of the other components of the device 100, etc.

Either one or both of the jaws' tissue engagement surfaces 18a, 18b can include one or more surface features thereon that are configured to help secure tissue grasped between the jaws 16a, 16b. For example, the one or more surface features can include a friction feature, such as teeth, ridges, or depressions, configured to increase friction between the grasped tissue and the surfaces 18a, 18b of the jaws 16a, 16b without tearing or otherwise damaging the tissue in contact with the one or more surface features. The one or more surface features can also be configured to facilitate the grasping tissue and forming substantially smooth, uniform layers of tissue to improve tissue effect. In this illustrated embodiment, one or more surface features in the form of a plurality of teeth 26 are positioned along an axial length of both of the engagement surfaces 18a, 18b.

The first and second jaws 16a, 16b can include features for interacting with a force-translating component, such a compression member, rod, or other structure extending through the shaft portion 12 and configured to effect at least one function of the end effector 14 such as closing, cutting tissue, etc. In the illustrated embodiment, the force-translating component is a compression member 28 configured to apply compressive forces on the jaws 16a, 16b and tissue grasped therebetween. In other embodiments, the force-translating component can be a rod or other structure capable of translating movement from the handle portion 10 to the end effector 14. The first and second jaws 16a, 16b can include first and second recessed slots (not shown, although they can be formed right at the cross section edge illustrated in FIG. 3) configured to receive portions 30a, 30b of the compression member 28 and to act as a track to direct movement of the compression member 28. As the compression member 28 is actuated to move distally along the axial length L_{A} of the jaws 16a, 16b, the compression member 28 is configured to apply a force to one or both of the jaws 16a, 16b to approximate their tissue engagement surfaces 18a, 18b closer together. In some embodiments, the compression member 28 can include a cutting member in the form of a cutting edge that is effective to transect tissue disposed within the jaws 16a, 16b as the compression member 28 is advanced distally. The cutting edge can be disposed on a distal most end 28d of the compression member 28, such as on a distal face of an upper flange 30a of the compression member 28 and/or being formed on the connecting portion 30c of the compression member. In some embodiments, the cutting member can be a knife blade or the like that is not attached to a compression member, such that the cutting member can advance and retract relative to the jaws 16a, 16b without applying compression to tissue grasped by the jaws 16a, 16b.

The compression member 28 can have various sizes, shapes, and configurations. As in this illustrated embodiment, the compression member 28 can have an elongate shape and can be configured to move proximally and distally along the end effector 14. The compression member 28 is this illustrated embodiment is at the distal end of the drive shaft 40 such that movement of the drive shaft 40 causes corresponding movement of the compression member 28, e.g., distal movement of the drive shaft 40 causes distal movement of the compression member 28. As shown in FIG. 4, the compression member 28 can have a proximal end 28p, a medial portion 28m, and a distal end 28d. A longitudinal axis L_{C} of the compression member 28 is substantially aligned and coaxial with longitudinal axis L₁ of the shaft 12, though other configurations are possible.

The compression member 28 is configured to be actuated from the proximal handle portion 10 of the instrument 100 by any suitable mechanism that is operatively coupled to the proximal end 28p of the compression member 28, such as via the closure actuator 20, as in this illustrated embodiment. The compression member 28 includes a connecting portion 30c and the upper and lower flanges 30a, 30b, thus providing an “I-beam” type cross-sectional shape of the compression member 28. In the illustrated embodiment, the upper and lower flanges 30a, 30b are positioned substantially perpendicular to the connecting portion 30c to form the “I-beam” shape. As previously mentioned, the upper and lower flanges 30a, 30b can be sized and shaped to slide in recessed slots in each of the upper and lower jaw 16a, 16b. This sliding contact of lateral edges of the flanges 30a, 30b and sides of each of the recessed slots may prevent lateral flexing of the jaws 16a, 16b.

The compression member 28 can have various other configurations. For example, the upper flange 30a can have a width that is greater than a width of the lower flange 30b, the widths being measured in a direction perpendicular to the longitudinal axis L_{C} of the compression member 28. For another example, the upper and lower flanges 30a, 30b can be disposed on or substantially on the distal end 28d of the compression member 28 and need not extend from the proximal end 28p to the distal end 28d of the compression member 28. For yet another example, the compression member 28 can be replaced more generally by a rod or inner shaft configured to advance distally to actuate the end effector 14.

The surgical device 100 is configured to apply energy to tissue disposed between the jaws 16a, 16b via at least one electrode located at the end effector 14 and associated with the jaws 16a, 16b. The surgical device 100 includes a firing actuator 24 configured to deliver energy to tissue grasped by the jaws 16a, 16b. Actuation of the firing actuator 24 configured to be actuated to complete a circuit to power the one or more electrodes associated with the jaws 16a, 16b (such as one or more electrodes on the engagement surfaces 18a, 18b of one or both of the jaws 16a, 16b) to seal tissue between the jaws 16a, 16b. More particularly, completion of the circuit by actuating the firing actuator 24 can allow electrical energy to pass from a power source 32 through one or more electrical leads 34 to at least one electrode 36 located at the end effector 14 that is configured to contact and delivery energy to tissue grasped by the end effector 14 to seal the tissue. Only the upper jaw 16a includes at least one electrode 36 in this illustrated embodiment, e.g., the at least one electrode 36 is disposed on the engagement surface 18a of the upper jaw 16a, but in other embodiments only the lower jaw 16b can include at least one electrode or each of the upper and lower jaws 16a, 16b can include at least one electrode. The power source 32 and the one or more electrical leads 34 are omitted from FIG. 1 for clarity of illustration. As shown in FIG. 3, the electrical lead(s) 34 extend through the inner passageway 38 along the shaft 12 to electrically connect the firing actuator 24 and the at least one electrode 36. The power source 32 is disposed in the housing 10 in this illustrated embodiment. In other embodiments the power source can be external of the housing 10, and the housing 10 can be configured to electrically connect to the power source, such as by way of a socket extending from the housing 10 to connect to the power source, e.g., by using a cord extending from the housing 10 or by using another connection. The cord can be feet long, e.g., ten feet or more, which is long enough to degrade signals and affect impedance measurement, as discussed herein.

The firing actuator 24 in this illustrated embodiment is in the form of a button but can have other configurations, e.g., a lever, a knob, etc. The firing actuator 24 can be configured to effect a function of the end effector 14 in addition to or instead of applying energy. By way of non-limiting example, the firing actuator 24 can be configured to be actuated to operate a cutting member to cut tissue grasped between the jaws 16a, 16b, such as a knife or other cutting member.

The electrode(s) 36 can have a variety of sizes, shapes, and configurations. As in this illustrated embodiment, the electrode 36 can be substantially flat and complementary to the substantially flat tissue-engaging surface 18a of the upper jaw 16a. Energy can be supplied thereto, for instance by the firing actuator 24, as described above. Other ways of energizing the electrode 36 (and other electrode(s) if more than one is provided and desired to be used, or the lower jaw's electrode if it includes the electrode instead of the upper jaw 16a) can be implemented, as will be appreciated by a person skilled in the art.

FIG. 3A illustrates another embodiment of a jaw 16′ that includes at least one electrode 36′. The jaw 16′ includes a plurality of longitudinally aligned electrodes 36a on each side of a central longitudinal slot 16s through which a cutting element is configured to translate, and the jaw 16′ includes an electrode 36b at a proximal tip of the jaw 16′. The jaw 16′ can be an upper jaw or a lower jaw.

FIG. 3B illustrates another embodiment of a jaw 16″ that includes at least one electrode 36″. The jaw 16″ includes a plurality of electrodes 36″ (five in this illustrated embodiment) arranged along a perimeter of the jaw 16″ so as to be arranged in an arc shape. The jaw 16″ can be an upper jaw or a lower jaw and is configured for use in an RF-only instrument or an instrument that uses RF and an additional modality.

FIG. 3C illustrates yet another embodiment of a jaw 16′″ that includes at least one electrode 36′″. The jaw 16′″ includes a plurality of longitudinally aligned electrodes 36c (four in this illustrated embodiment) and a proximal tip electrode 36d that are arranged along a perimeter of the jaw 16′″ so as to be arranged in an arc shape. FIG. 3C also shows for each of the four longitudinally aligned electrodes 36c a wire 34′″ or electrical lead operative coupled thereto. The jaw 16′″ can be an upper jaw or a lower jaw.

FIGS. 3D-3F illustrate another embodiment of an upper jaw 16a′. The upper jaw 16a′ includes an inner electrode 36i, an outer electrode 36o, insulation 37 positioned between the inner and outer electrodes 36i, 36o, and an electrical lead 34e operatively coupled to a lead interface 35 for the electrodes 36i, 36o. The upper jaw 16a′ is configured for use in surgical instrument configured to deliver RF and ultrasonic energy.

Referring again to FIGS. 1-3, the device 100 includes a motor 42 and a controller 44 each disposed within the housing 10. The motor 42 in this illustrated embodiment is operatively coupled to the drive shaft 40 and the compression member 28, such as via a gear and rack. Activation of the motor 42, e.g., by actuating the closure actuator 20, can thus be configured to advance and/or retract the compression member 28. The controller 44 can be configured to operatively couple the closure actuator 20 and the motor 42 such that actuation of the closure actuator 20 causes activation of the motor 42 in response to control signals transmitted to the motor 42 from the controller 44. The controller 44 can be configured to operatively couple the firing actuator 24 and the power source 32 such that actuation of the firing actuator 24 causes energy application. The motor 42, power source 32, and controller 44 can be disposed at various locations in the device 100, such as in the proximal handle portion 10, although any one or more of the motor 42, power source 32, and controller 44 can be located off-board of the device 100 and operatively coupled thereto, such as with a cord or other wired or wireless connection. In other embodiments, the surgical device can lack the motor, power source, and controller such that end effector opening/closing and compression member advancement/retraction can be manually accomplished.

Exemplary embodiments of devices and methods for grasping and sealing tissue are further described in U.S. Pat. No. 9,585,715 entitled “Electrosurgical Sealing And Transecting Devices And Methods With Improved Application Of Compressive Force” filed Jan. 7, 2014, U.S. Pat. Pub. No. 2013/0161374 entitled “Layer Arrangements For Surgical Staple Cartridges” filed Feb. 8, 2013, U.S. Pat. No. 8,888,809 entitled “Surgical Instrument With Jaw Member” filed Oct. 1, 2010, and U.S. Pat. No. 6,978,921 entitled “Surgical Stapling Instrument Incorporating An E-Beam Firing Mechanism” filed May 20, 2003, which are incorporated by reference herein in their entireties.

The device 100 is configured to operatively connect to a generator 46, shown in FIGS. 2 and 5, to provide an off-board power source for powering one or more components of the device 100, such as powering the motor 42 and/or powering the electrode 36 as an alternate or in addition to the on-board power source 32. In the illustrated embodiment, the generator 46 is configured to operatively couple to the firing actuator 24. The generator 46 can have a variety of configurations, such as a radiofrequency (RF) generator. The generator 46 being a separate unit from the device 100 that is configured to electrically connect to the device 100 may allow a weight and size profile of the device 100 to be reduced, may allow different types of generators to be operatively coupled to the device 100, e.g., to allow users to select an appropriate generator for a particular procedure, may facilitate repair and/or upgrade of generators, and/or may reduce overall cost of the device 100. The generator 46 can include at least one port configured to physically connect to the surgical device 100 or other surgical device, such as by a cord of the surgical device plugging into the port.

As in this illustrated embodiment, the generator 46 can include an energy application module 48, a signal transmission module 50, a signal receipt module 52, and an analyzer module 54. The modules 48, 50, 52, 54 can each be part of a computer system at the generator 46. The computer system can include one or more processors which can control the operation of the computer system. The processor(s) can include any type of microprocessor or central processing unit (CPU), including programmable general-purpose or special-purpose microprocessors and/or any one of a variety of proprietary or commercially available single or multi-processor systems. The computer system can also include one or more memories, which can provide temporary storage for code to be executed by the processor(s) or for data acquired from one or more users, storage devices, and/or databases. The memory can include read-only memory (ROM), flash memory, one or more varieties of random access memory (RAM) (e.g., static RAM (SRAM), dynamic RAM (DRAM), or synchronous DRAM (SDRAM)), and/or a combination of memory technologies. The various elements of the computer system can be coupled to a bus system. The computer system can also include one or more network interface(s), one or more input/output (IO) interface(s), and one or more storage device(s). A computer system can also include any of a variety of other software and/or hardware components, including by way of non-limiting example, operating systems and database management systems. Although an exemplary computer system is described herein, it will be appreciated that this is for sake of generality and convenience. In other embodiments, the computer system may differ in architecture and operation from that shown and described here.

The energy application module 48 is configured to deliver energy to a surgical device connected thereto to allow the surgical device's one or more electrodes to apply energy to matter in contact therewith, e.g., to allow the electrode(s) 36 of the device 100 to apply energy. The energy application module 48 can thus be configured to operatively couple to the lead(s) 34 of the surgical device 100 such that the lead(s) 34 carry energy supplied by the generator 46 to the electrode(s) 36.

The signal transmission module 50 is configured to transmit electrical signals to the surgical device 100 or other surgical device connected to the generator 46. The electrical signals can be transmitted along the device's lead(s) 34. The device 100 can include four leads 34, e.g., first, second, third, and fourth wires, that each carry an electrical signal to/from the generator 46, as discussed herein. FIG. 3C illustrates one embodiment in which a surgical device includes four leads. As discussed above, the electrical signals can have a variety of configurations. For example, the four wires can carry two V+ signals and two V− signals. For another example, each of the four wires can carry a signal having a different low frequency in a range of about 500 to 2000 Hz. Surgical RF generators have a blocking capacitor per government safety regulations to ensure that no direct current (DC) components resulting from source (generator) faults and/or from tissue partially rectifying the alternating current (AC) of the source's RF output are present and delivered to the patient. The value of the blocking capacitor is a known value: 0.047 μF in the U.S. per FDA regulations. Thus, the generator 46 can include a blocking capacitor in series with RF output. Due to the presence of the blocking capacitor, impedance at low frequencies within the range of about 500 to 2000 Hz will not be accurate because the blocking capacitor will appear as a resistor and introduce resistance. Thus, the generator can include at least one element that bypasses the blocking capacitor for purposes of impedance measurement. In this way, any DC offset from the patient will not go back to the patient when energy is delivered thereto via the surgical device's electrode(s). The at least one bypass element can have a variety of configurations, such as relay contacts, two MOSFET transistor in an AC-switch arrangement, or two silicon controlled rectifiers (SCRs) in a parallel AC-switch arrangement.

The signal receipt module 52 is configured to receive return signals from the surgical device 100 or other surgical device connected to the generator 46 in response to the transmitted electrical signals. The return signals can be transmitted along the device's lead(s) 34.

The analyzer module 54 is configured to analyze the received return signals to determine an impedance of the matter in contact with the electrodes(s) 36 to which the electrical signals were transmitted. Various values of impedance can be encountered during use of the surgical device 100 and other advanced bipolar surgical devices and advanced ultrasonic surgical devices during performance of different functions, such as coagulation, debulking, feathering, vessel sealing, spot coagulation, tissue transection, etc. Impedances may be measured in a very wide dynamic range, e.g., in a range of about 2 Ohms to 3000 Ohms, during various uses of surgical devices such that timely and accurate measurement of the impedance when energy is being applied or is about to be applied may significantly affect appropriate, safe energy delivery. For example, lower impedance tissues such as vessels, connective tissues, etc. can be encountered, e.g., by larger jaw surgical devices such as the ENSEAL® X1 Large Jaw available from Ethicon, LLC of Cincinnati, Ohio and the LigaSure Impact™ available from Medtronic, Inc. of Minneapolis, Minn., with impedance being measured in a range from about 2 Ohms to 200 Ohms or about 3 Ohms to 200 Ohms. Such surgical devices, including the surgical device 100 if so configured, can be configured to terminate tissue treatment when the measured impedance is greater than a predetermined upper limit, such as the high end of the range, e.g., 200 Ohms, or a value above the high end of the range, e.g., 500 Ohms when the high end of the range is 200 Ohms. The predetermined upper limit can depend on, for example, the particular algorithm used by the surgical device and generator. For another example, higher impedance tissues can be encountered, e.g., by smaller jaw surgical devices such as the ENSEAL® G2 Articulating Tissue Sealers available from Ethicon, LLC of Cincinnati, Ohio and the LigaSure Small Jaw™ available from Medtronic, Inc. of Minneapolis, Minn., with impedance being measured in a range from about 20 Ohms to 400 Ohms. Such surgical devices, including the surgical device 100 if so configured, can be configured to terminate tissue treatment when the measured impedance is greater than a predetermined upper limit, such as the high end of the range, e.g., 400 Ohms, or a value above the high end of the range, e.g., 500 Ohms when the high end of the range is 400 Ohms. The predetermined upper limit can depend on, for example, the particular algorithm used by the surgical device and generator. For yet another example, impedance can be measured in some instances as high as about 3000 Ohms or greater.

The analyzer module 54 is also configured to determine based on the determined impedance the energy for the energy application module 48 to deliver to the surgical device 100 or other surgical device connected to the generator 46. The energy can be determined in a variety of ways.

For example, the analyzer module 54 can be configured to look up the determined impedance in a table, such as in a predetermined lookup table stored in the generator's memory, that correlates impedances with energies. The energy that corresponds to the determined impedance can be the energy delivered by the energy application module 48. As mentioned above, in most instances, the matter contacted by the electrodes(s) 36 will be tissue, in which case energy may be applied thereto via the surgical device's electrode(s) 36. In other instances, the matter contacted by the electrodes(s) 36 will not be tissue, in which case energy should not be applied to the matter contacted by the electrodes(s) 36. Thus, if the determined impedance is not in the table, the analyzer module 54 can be configured to determine that the matter in contact with the electrode(s) 36 is not tissue and that an error condition is therefore present. In response to determining that an error condition is present, the analyzer module 54 can be configured to cause an alert to be provided. The alert can be provided in any one or more ways, such as via a speaker that is part of the generator 46 or other computer system, via a display screen of the generator 46 or other computer system, via shaking of the surgical device 100, etc. The table that correlates impedances with energies can correlate impedance values with algorithms to be executed by the analyzer module 46 such that looking up the determined impedance in the table identifies an algorithm according to which energy should be delivered by the energy application module 48. The algorithm can reflect electrical characteristics, such as voltage, current, and power, to be used in delivering the energy. Various embodiments of algorithms are further discussed in U.S. Pat. No. 9,737,355 entitled “Controlling Impedance Rise In Electrosurgical Medical Devices” filed Mar. 31, 2014, which is hereby incorporated by reference in its entirety.

For another example, the analyzer module 54 can be configured to dynamically determine, using the determined impedance, an algorithm according to which energy should be delivered by the energy application module 48. In at least some embodiments, a hybrid approach can be used in which the analyzer module 54 looks up the determined impedance in a table that correlates impedances with energies and that energy is initially used by the energy application module 48, and the analyzer module 54 is configured to dynamically switch between one or more other algorithms based on later-determined impedances. In other words, the signal transmission module 50 can be configured to send a sequential series of electrical signals to the surgical device 100 (or other surgical device connected to the generator 46), the signal receipt module 52 can be configured to receive a sequential series of return signals in response and determine an impedance, and the analyzer module 54 can be configured to look up an algorithm corresponding to the most recently determined impedance and use that algorithm. Dynamically changing algorithms to deliver energy during the application of energy by the electrode(s) of the surgical device 100 (or other surgical device connected to the generator 46) may provide for a better seal of tissue and/or may account for moisture at the electrode(s) that dissipates or evaporates during the energy application.

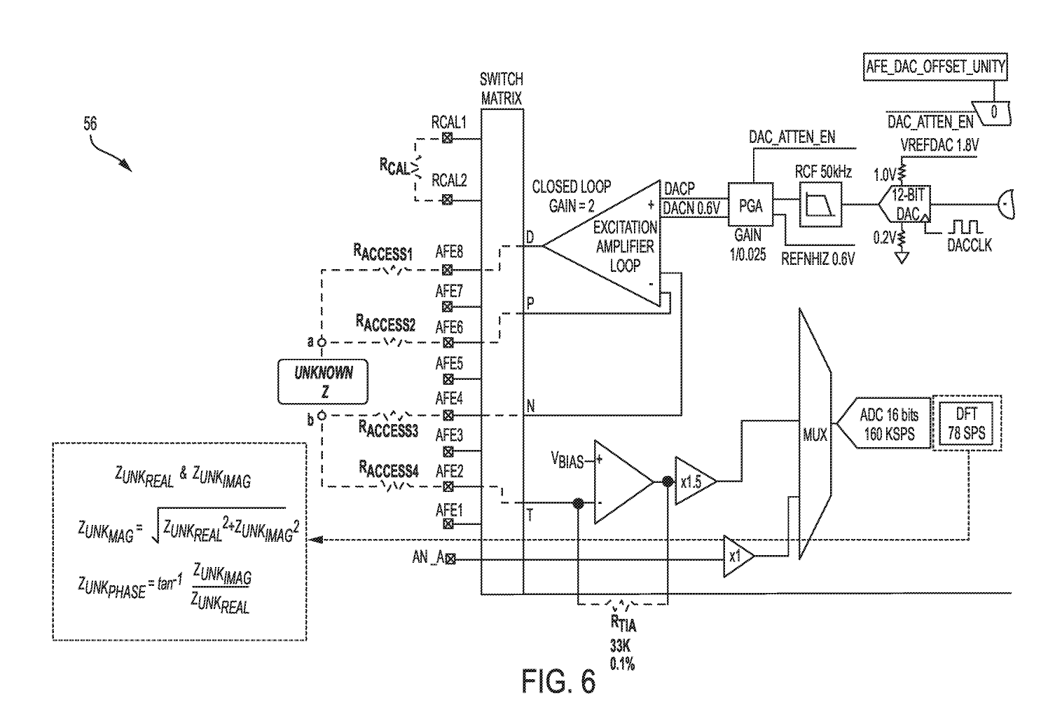

FIG. 6 illustrates one embodiment of circuitry 56 that the generator 46 can include and use to measure impedance using a four-wire architecture. The circuitry 56 of FIG. 6 can be an ADuCM350 or AD5940 system available from Analog Devices, Inc. of Norwood, Mass. and is further described in AN-1302 (Application Note), “Optimizing the ADuCM350 for 4-Wire-Bio-Isolated Impedance Measurement Applications,” 2013. In general, the circuitry 56 is configured to measure impedance (labeled “Unknown Z” in FIG. 6) using four wires (the four lines in FIG. 6 including RACCESS resistors).

The energy output signal from the generator 46 can in some instances cause damage to the circuitry 56 unless the circuitry 56 is isolated. Isolation can be achieved in a variety of ways, such as by using relays to disengage the circuitry from the load, a protection circuit on each of the four wires (e.g., similar to protection provided for ECG/EKG machines to prevent damaging the machine's circuitry when defibrillating a patient), an element to prevent RF energy from damaging the circuitry 56, etc.

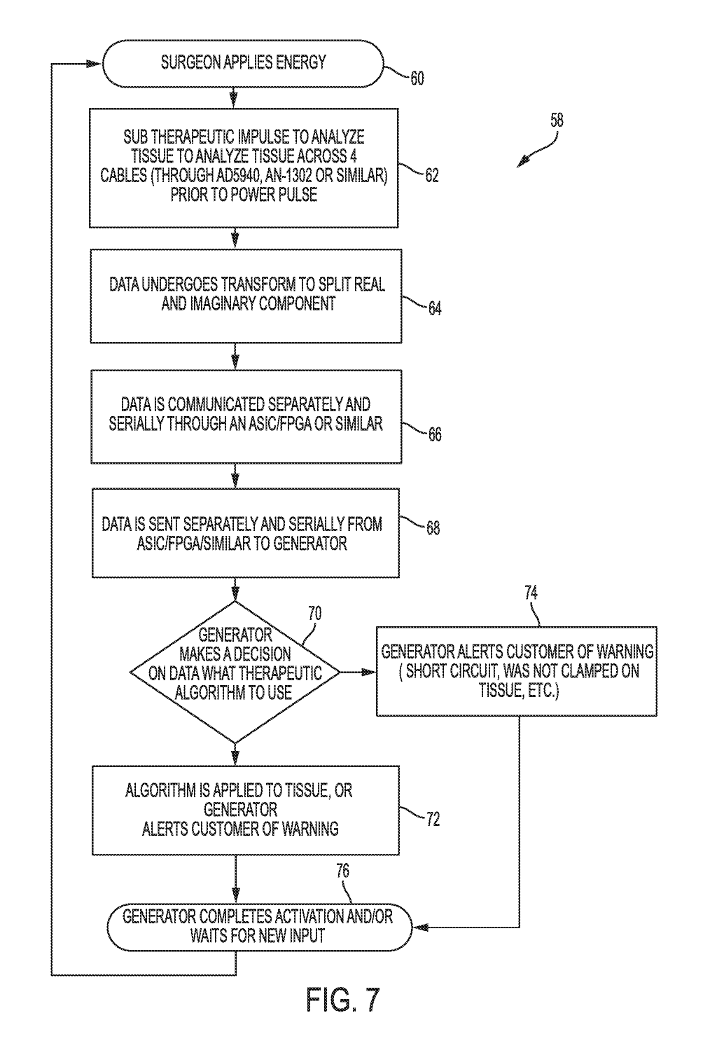

One embodiment of a method 58 of measuring impedance for an electrosurgical tool is illustrated in FIG. 7. Although the method 58 of FIG. 7 is discussed with respect to the surgical device 100 and generator 46 of FIGS. 1-3, any of the surgical devices and generators described herein can be similarly used.

In the method 78, the device 100 is connected to the generator 46, such as by removably and replaceably coupling the device 100 to the generator 46 by plugging a cord of the device 100 into a port of the generator 46. In other embodiments, as mentioned above, the device 100 can connect to the generator 46 in other ways, or the generator 46 can be on board the device 100.

With the device 100 connected to the generator 46, a surgeon or other user can apply 60 energy using the surgical device 100 by actuating the firing actuator 24. In response to the actuation of the firing actuator 24, impedance is measured prior to energy being applied via the electrode(s) 36 in order to determine the energy to apply, as discussed herein. In particular, the actuation of the firing actuator 24 can cause a signal to be transmitted from the surgical device 100 to the generator 46, e.g., to the energy application module 48. In response to the receipt of the signal indicative of actuation of the firing actuator 24, an electrical signal can be transmitted 62 from the generator 46, e.g., from the signal transmission module 50 thereof, along four wires to the surgical device 100, e.g., along the lead(s) 34 to the electrode(s) 36. The generator 46, e.g., the signal transmission module 50 thereof, can split 64 the electrical signal into real and imaginary components for transmission along the four wires, e.g., two V+ signals and two V− signals. The electrical signal can then be communicated 66 to the surgical device 100, e.g., to the controller 44 thereof that can include a field-programmable array (FGPA), microprocessor, an application-specific integrated circuit (ASIC), or the like to facilitate transmission of signals to/from the generator 46. The controller 44 can be configured to transmit the electrical signal along the lead(s) 34 to the electrode(s) 36. Noise filtering may be used to reduce or prevent noise in the signal transmission, such as via software executed by the controller 44 is the signal is not saturated, via hardware in the generator 46 or the surgical device 100, or via filtering/noise isolation provided by shielding through the two pairs of wires, first or second order filters, a tank circuit on the lead(s) 34, etc. In response, a return electrical signal can be communicated 68 from the surgical device 100, e.g., from the controller 44 that receives data from the electrode(s) 36 along the lead(s) 34, to the generator 46, e.g., to the signal receipt module 52. Noise filtering may similarly be used on the return signal. The generator 46 can be configured to analyze, as described herein, the received return signal to determine 70 whether to deliver 72 energy to the surgical device 100 or to cause 74 an alert of an error condition to be provided. After energy is delivered 72 or after the alert is caused 74 to be provided, the generator 46 completes activation (e.g., completes energy delivery or alert provision) and awaits for new input, e.g., awaits for another actuation of the firing actuator 24.

Some surgical instruments, so-called combined ultrasonic and RF surgical devices, are configured to deliver ultrasonic energy and RF energy. In these instruments, discrepancies on delivering power under the correct conditions exist for different types of tissue. On larger vessels, at a clamp force that is appropriate for ultrasonic seal and cut, RF does not typically result in good seals without adding ultrasonic energy. A conflict exists where the added ultrasonic power applied during seal only has a potential to cut thinner tissue. This conflict may be addressed by detecting tissue size and using the detected tissue size to regulate an amount of ultrasonic power applied during a seal-only operation of the device, e.g., during sealing of the tissue when cutting of the tissue is not occurring. For example, using the device 100 of FIG. 1 by way of example only, the controller 44 can be configured to receive an input indicated of a size of tissue clamped by the jaws 16a, 16b of the end effector 14 and use the input to determine an amount of ultrasonic energy to apply to the clamped tissue during a seal-only operation of the device 100.

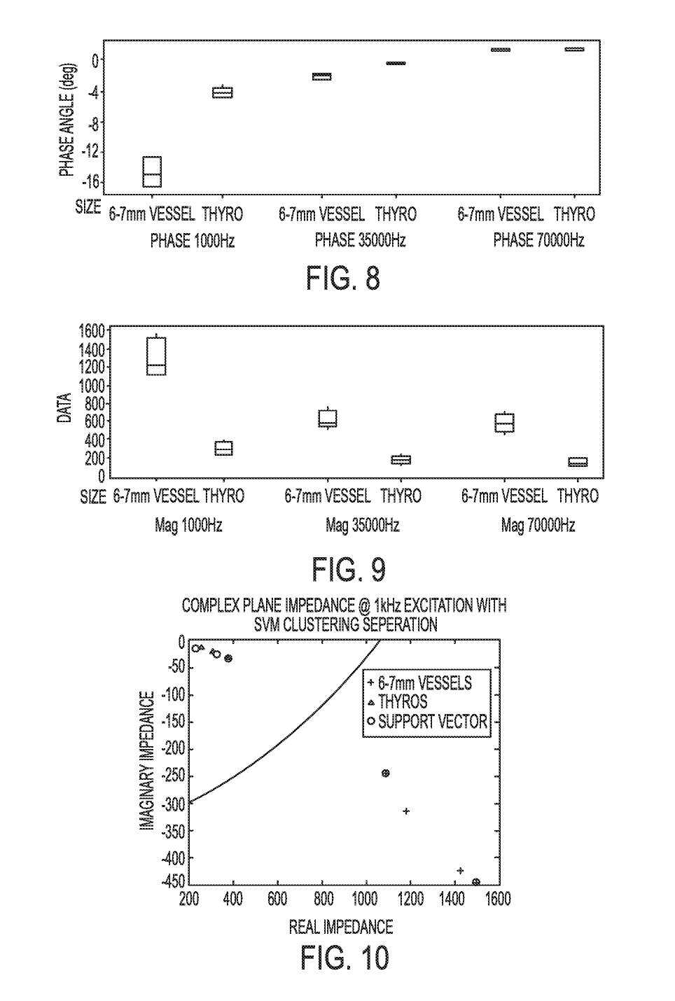

Tissue size can be detected by measuring impedance of the tissue, such as by using the techniques discussed herein. As shown in the boxplot graphs of FIGS. 8 and 9, tissue impedance is strongly correlated to the amount of tissue present in jaws of a surgical device, e.g., by the amount of tissue clamped between the jaws. FIG. 8 shows a phase angle comparison of different size vessels at various frequencies, and FIG. 9 shows a magnitude comparison of the different size vessels at various frequencies. FIGS. 8 and 9 indicate that the correlation between and impedance and amount of tissue is more evident at lower frequency excitation. FIG. 10 shows complex impedance measure at 1000 Hz of excitation on large (6-7 mm) vessels and small (2-4 mm) thyrocervical (thyros) vessels. The plot line indicates the result of training a support vector machine for classification. FIG. 10 indicates that the value from a low frequency impedance measure can be used to detect tissue size. The detected tissue size may then be used to regulate an amount of ultrasonic power to be used in a seal-only combined ultrasonic/RF algorithm, optimal RF control, and actuated clamp force.

The sensitivity of RF tissue impedance to power input is high in post bathtub RF tissue sealing. During the bathtub, large amounts of power can be delivered with little or no effect on tissue impedance. Accordingly, it can be beneficial to apply a high level of ultrasonic power during the RF bathtub, which results in additional heat from ultrasonic power. To prevent the ultrasonic energy from cutting of the tissue, ultrasonic power can be turned lower or off at a point during sealing of the tissue. Since exiting the bathtub is a good indicator that tissue has sealed, RF impedance is a good indicator of when to reduce ultrasonic power (either by lowering the ultrasonic power or by turning ultrasonic power off). The following hyperbolic tangent mixing function equation can be used to regulate ultrasonic power as a function of RF impedance, where Iₕ is the applied ultrasonic power, Iₕₘᵢₙ is the lower level of post bathtub ultrasonic power that is to be delivered, Iₕₘₐₓ is the higher level of bathtub ultrasonic power that is to be delivered, m is the rate at which the ultrasonic power is to decay as a function of RF impedance, Zₜᵣₐₙₛᵢₜᵢₒₙ is the RF impedance when the ultrasonic power is at 50% of its range, and Z is the tissue impedance measurement.

$${I}_{h} = ( \frac{{I}_{hmin} - {I}_{hmax}}{2} ) \cdot ( tanh ( m \cdot Z - m \cdot {Z}_{transition} ) - 1 ) + {I}_{hmin}$$

Parameters in the hyperbolic tangent mixing function equation can be selected based on the complex impedance measure since the parameters can vary based on the amount of tissue clamped by the jaws. The variables in the hyperbolic tangent mixing function equation to be optimized are Iₕₘᵢₙ, Iₕₘₐₓ, m, and Zₜᵣₐₙₛᵢₜᵢₒₙ. These ultrasonic optimization variables (Iₕₘᵢₙ, Iₕₘₐₓ, m, and Zₜᵣₐₙₛᵢₜᵢₒₙ) can be optimized for different sizes of tissue to achieve seal only without cutting. The variable Iₕₘₐₓ will be higher for thicker tissues to promote sealing and lower for thinner tissues to prevent cutting. Based on complex impedance measures, the ultrasonic optimization variables that are best suited to that tissue size can be implemented. The value of these variables for different tissue sizes can be predetermined by benchtop and in vivo testing and preprogrammed into the controller for the source of the power, the surgical device or the generator. Different tissue sizes and types may thus have different ultrasonic optimization variables for optimal performance.

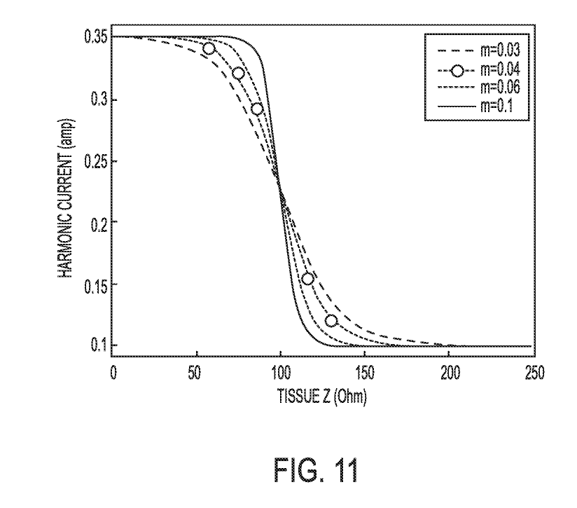

FIG. 11 illustrates one example of ultrasonic power as a function of RF impedance for specified variables and different values of m in the hyperbolic tangent mixing function equation. In FIG. 11, Iₕ is plotted on they axis, Z is plotted on the x axis, Iₕₘᵢₙ equals 0.1 Amps, Iₕₘₐₓ equals 0.35 Amps, m has various values as shown in the legend of FIG. 11, and Zₜᵣₐₙₛᵢₜᵢₒₙ equals 100 Ohms.

In a surgical device with actuated clamp force, such as powered surgical devices and robotically controlled surgical devices, the clamp force can be modulated and optimized to match the bite size, e.g., the amount of tissue being clamped by the device's jaws. An optimal clamp force exists with different size of tissue bites. Actuating clamp force based on an initial or real time complex impedance sweep to determine the best clamp force to apply would result in optimal sealing and prevent cutting.

A person skilled in the art will appreciate that the devices, systems, and methods disclosed herein have application in conventional minimally-invasive and open surgical instrumentation as well application in robotic-assisted surgery. In some embodiments, the devices, systems, and methods described herein are provided for open surgical procedures, and in other embodiments, the devices, systems, and methods are provided for laparoscopic, endoscopic, and other minimally invasive surgical procedures. The devices may be fired directly by a human user or remotely under the direct control of a robot or similar manipulation tool. However, a person skilled in the art will appreciate that the various methods, systems, and devices disclosed herein can be used in numerous surgical procedures and applications. Those skilled in the art will further appreciate that the various instruments disclosed herein can be inserted into a body in any way, such as through a natural orifice, through an incision or puncture hole formed in tissue, or through an access device, such as a trocar cannula. For example, the working portions or end effector portions of the instruments can be inserted directly into a patient's body or can be inserted through an access device that has a working channel through which the end effector and elongated shaft of a surgical instrument can be advanced.

The devices disclosed herein can be designed to be disposed of after a single use, or they can be designed to be used multiple times. In either case, however, the device can be reconditioned for reuse after at least one use. Reconditioning can include any combination of the steps of disassembly of the device, followed by cleaning or replacement of particular pieces and subsequent reassembly. In particular, the device can be disassembled, and any number of the particular pieces or parts of the device can be selectively replaced or removed in any combination. Upon cleaning and/or replacement of particular parts, the device can be reassembled for subsequent use either at a reconditioning facility, or by a surgical team immediately prior to a surgical procedure. Those skilled in the art will appreciate that reconditioning of a device can utilize a variety of techniques for disassembly, cleaning/replacement, and reassembly. Use of such techniques, and the resulting reconditioned device, are all within the scope of the present application.

One skilled in the art will appreciate further features and advantages of the invention based on the above-described embodiments. Accordingly, the invention is not to be limited by what has been particularly shown and described, except as indicated by the appended claims. All publications and references cited herein are expressly incorporated herein by reference in their entirety.

What is claimed is:

1. A surgical system, comprising:
a generator configured to
transmit a signal along first, second, third, and fourth wires to an end effector of a surgical device in contact with matter within a body of a patient,
receive a return signal from each of the first, second, third, and fourth wires,
analyze the return signals to determine an impedance of the matter, and
determine, based on the determined impedance, an algorithm for application of energy from the generator to the matter via an electrode at the end effector of the surgical device in contact with the matter.

2. The system of claim 1, wherein the matter is tissue, and the generator is configured to apply the energy to the matter using the determined algorithm.

3. The system of claim 1, wherein the generator is configured to determine, based on the determined impedance, that an error condition is present, and the generator is configured to cause an alert to be provided to a user that indicates the presence of the error condition, wherein the error condition is one of a short circuit, an open circuit, and the determined impedance indicating that the matter is not tissue.

4. The system of claim 1, wherein determining the impedance of the matter includes determining a real component of the impedance and an imaginary component of the impedance to determine a phase shift, and using the determined phase shift to identify actual impedance of the matter.

5. The system of claim 1, wherein the matter is tissue, determining the impedance of the matter indicates a type of the tissue, and determining the algorithm includes selecting one of a plurality of predetermined algorithms based on the type of the tissue.

6. The system of claim 1, wherein the matter is tissue, determining the impedance of the matter indicates a type of the tissue, and determining the algorithm includes dynamically generating an algorithm based on the type of the tissue.

7. The system of claim 1, wherein the transmitted signal is an excitation signal including a positive supply voltage along the first and third wires and a negative supply voltage along the second and fourth wires.

8. The system of claim 1, wherein the transmitted signal is a drive signal having a frequency in a range of about 500 to 2000 Hz.

9. The system of claim 1, further comprising the surgical device.

10. A surgical system, comprising:
a surgical device including a proximal handle portion having an elongate shaft extending distally therefrom, the elongate shaft having an end effector at a distal end thereof, the end effector including an electrode configured to contact tissue and apply energy thereto, and the surgical device being configured to
receive from a generator a first signal along first, second, third, and fourth wires that extend to the end effector,
in response to receiving the first signal, return a second signal to the generator along the first, second, third, and fourth wires, and
after returning the second signal, receive a third signal from the generator and in response thereto apply the energy via the electrode.

11. The system of claim 10, further comprising the generator;
wherein the generator is configured to receive the second signal and in response thereto determine an impedance of the tissue contacted by the electrode, and the third signal is determined in real time by the generator based on the determined impedance.

12. A surgical method, comprising:
transmitting a signal along each of first, second, third, and fourth wires from a generator to a distal end of a surgical device in contact with matter within a body of a patient;
receiving at the generator a return signal from each of the first, second, third, and fourth wires;
analyzing the return signals to determine an impedance of the matter; and
determining, based on the determined impedance, an algorithm for application of energy from the generator to the matter via an electrode at the distal end of the surgical device in contact with the matter.

13. The method of claim 12, further comprising applying the energy from the generator to the matter using the determined algorithm, wherein the matter is tissue.

14. The method of claim 12, further comprising determining, based on the determined impedance, that an error condition is present; and
causing an alert to be provided to a user that indicates the presence of the error condition, wherein the error condition is one of a short circuit, an open circuit, and the determined impedance indicating that the matter is not tissue.

15. The method of claim 12, wherein determining the impedance of the matter includes determining a real component of the impedance and an imaginary component of the impedance to determine a phase shift, and using the determined phase shift to identify actual impedance of the matter.

16. The method of claim 12, wherein the matter is tissue, determining the impedance of the matter indicates a type of the tissue, and determining the algorithm includes selecting one of a plurality of predetermined algorithms based on the type of the tissue.

17. The method of claim 12, wherein the matter is tissue, determining the impedance of the matter indicates a type of the tissue, and determining the algorithm includes dynamically generating an algorithm based on the type of the tissue.

18. The method of claim 12, wherein the transmitted signal is an excitation signal including a positive supply voltage along the first and third wires and a negative supply voltage along the second and fourth wires.

19. The method of claim 12, wherein the transmitted signal is a drive signal having a frequency in a range of about 500 to 2000 Hz.

20. The method of claim 12, further comprising removably and replaceably connecting the surgical device to the generator.
